SEQUENCE LISTING (18 sequences)

SEQ ID NO: 1 - DNA, 25 bp, Artificial Sequence
  Synthetic
gcctctagactcatagaacttagtc

SEQ ID NO: 2 - DNA, 22 bp, Artificial Sequence
  Synthetic
gtgaggtcaggccattgcattg

SEQ ID NO: 3 - DNA, 30 bp, Artificial Sequence
  Synthetic
atggttttagaaatcatcaagcctagggca

SEQ ID NO: 4 - DNA, 30 bp, Artificial Sequence
  Synthetic
aatgaaaggaggggaggatgacagaaatgt

SEQ ID NO: 5 - DNA, 20 bp, Artificial Sequence
  Synthetic
gttgtcatcagactttgacc

SEQ ID NO: 6 - DNA, 20 bp, Artificial Sequence
  Synthetic
ttcagttcatatggaccaga

SEQ ID NO: 7 - DNA, 20 bp, Artificial Sequence
  Synthetic
tggcattgatctggttcatc

SEQ ID NO: 8 - DNA, 20 bp, Artificial Sequence
  Synthetic
gtttaggaatcttcccactt

SEQ ID NO: 9 - DNA, 17 bp, Artificial Sequence
  Synthetic
cccctcagcaacactcc

SEQ ID NO: 10 - DNA, 17 bp, Artificial Sequence
  Synthetic
ggtcagaagggcagaga

SEQ ID NO: 11 - DNA, 23 bp, Artificial Sequence
  Synthetic
ttggaggatggcccatgaagacc

SEQ ID NO: 12 - DNA, 23 bp, Artificial Sequence
  Synthetic
ctgttacgcgcccggatgaaaaa

SEQ ID NO: 13 - DNA, 19 bp, Artificial Sequence
  Synthetic
tcgtcactctcctggtcag

SEQ ID NO: 14 - DNA, 18 bp, Artificial Sequence
  Synthetic
cacgcatgtgacctcagg

SEQ ID NO: 15 - DNA, 18 bp, Artificial Sequence
  Synthetic
tcacatgcgtggtggtgg

SEQ ID NO: 16 - DNA, 20 bp, Artificial Sequence
  Synthetic
tggaggtgcataatgccaag

SEQ ID NO: 17 - DNA, 20 bp, Artificial Sequence
  Synthetic
tggaggtgcataatgccaag

SEQ ID NO: 18 - DNA, 19 bp, Artificial Sequence
  Synthetic
agaagacgttcccctgctg

(19) United States
(12) Patent Application Publication
(10) Pub. No.: US 2004/0229228 A1
(43) Pub. Date: Nov. 18, 2004

(54) Il-1 genotype in early kidney allograft rejection

(75) Inventors: John Ernest Sims, Seattle, WA (US); Dixon B. Kaufman, Chicago, IL (US)

Correspondence Address: MEDLEN & CARROLL, LLP, Suite 350, 101 Howard Street, San Francisco, CA, 94105, US

(73) Assignees: Northwestern University, Evanston, IL; Amgen, Inc., Seattle, WA

(21) Appl. No.: 10/626,830

(22) Filed: Jul. 24, 2003

Related U.S. Application Data
(60) Provisional application No. 60/398,986, filed on Jul. 25, 2002.

(51) Int. Cl. C12Q 1/68

(52) U.S. Cl. 435/6

(57) ABSTRACT

There are disclosed methods for identifying patients at increased risk for early rejection of a kidney allograft, as well as primer sets and kits useful in such methods. Also disclosed are methods of treating allograft recipients using immunosuppressive therapy, based on their risk for early kidney graft rejection.

The present application claims priority to U.S. Provisional Patent Application No. 60/398,986, filed Jul. 25, 2002, the disclosure of which is herein incorporated by reference in its entirety.

### FIELD OF THE INVENTION

The present invention relates generally to the field of genotyping, and in particular to determining the genotype of an individual for an IL-1 family member.

### BACKGROUND OF THE INVENTION

The IL-1 family is a large group of structurally related cytokines whose primary function is to modulate immune and inflammatory responses. There are ten known IL-1 family members which include IL-1 alpha (IL-1F1), IL-1 beta (IL-1F2), IL-1 receptor antagonist (IL-1ra; IL-1F3), IL-18 (IGIF; IL-1F4), IL-1F5 (IL-1 delta, IL-1Hy1), IL-1F6 (IL-1 epsilon), IL-1F7 (IL-1 zeta, IL-1H4, IL-1RP1, or IL-1H), IL-1F8 (IL-1eta, IL-1H2), IL-1F9 (IL-1H1, IL-1RP2, IL-1 epsilon) and IL-1F10 (IL-1Hy2, FSKG75) (Sims et al., Trends Immunol. 22:536, 2001). The genes for several IL-1 family members (IL-1alpha, IL-1beta and IL-1ra; the corresponding genes are referred to as IL1A, IL1B, and IL1RN) map to chromosome 2 (2q13; Nicklin, et al., Genomics 19:382, 1994).

Polymorphisms in the IL-1 gene complex have been associated with several disease states, including osteoporosis (U.S. Pat. No. 5,698,399), periodontal disease (U.S. Pat. No. 5,686,246), diabetic nephropathy (Blakemore, et al., Hum. Genet 97:369, 1996), alopecia areata (Cork et al., Dermatol Clin 14:671, 1996), acute graft-versus-host disease in bone marrow transplants (Cullup et al., Br. J. Haematol 113:807, 2001), and other, inflammatory or autoimmune conditions (see U.S. Pat. No. 6,268,142). Moreover, a rare combination of alleles in this gene complex has been associated with the presence of detectable levels of IL-1 beta in plasma from healthy individuals (Hulkkonen et al., Eur. Cytokine Netw. 11:251, 2001).

End stage renal disease (ESRD) is a condition in which chronic renal failure has lead to irreversible loss of kidney function to a degree that requires either dialysis or kidney transplant to avoid uremia. Although dialysis is capable of prolonging the lives of ESRD patients for years, organ transplant is the preferred treatment despite the inherent challenges in identifying suitable organs and maintaining the functionality of the transplanted kidney. The major risk associated with kidney allografts is rejection of the transplanted kidney, either acute or chronic. Acute rejection is immunologically mediated, and involves primarily the activation of T lymphocytes; chronic rejection is less well understood, but may also involve non-immunologic factors such as the age, number of nephrons and ischemic history of the donor kidney.

The immunologic basis of acute renal transplant rejection is complex, and may involve humoral as well as cellular aspects of an immune response; the type and nature of the response depends on the genetic differences between the donor and the recipient, and presensitization of the donor, among other factors. Because acute rejection is immunologically mediated, various immunosuppressive therapies have been used to enhance graft survival, including treatment with corticosteroids, antimetabolites, cytotoxic agents, and cyclosporine. However, such immunosuppressive therapies result in generalized immunosuppression, leading to increased risk for infectious or lymphoproliferative diseases.

One aspect of renal transplantation that is currently the subject of interest is the relationship (or lack thereof) of genetic variations in genes of immunological importance (i.e., cytokines, chemokines, and their receptors; Suthanthiran, M., Curr. Opin. Urol. 10:7, 2000) to graft rejection. Polymorphisms in the genes for Interleukin-10 (IL-10), interferon-gamma (IFN-gamma), and tumor necrosis factor alpha (TNF-alpha) have been associated with acute renal allograft rejection (Sankaran et al., Kidney Int. 56:1, 1999; Asderakis et al., Transplantation 15:674, 2001). However, no association has been shown between early acute rejection of a kidney allograft (defined as rejection that occurs within three weeks of transplant) and genetic variations in immunologically important molecules. The ability to identify patient populations that are at increased risk for early kidney allograft rejection will facilitate development of immunosuppressive therapies that can be tailored to the needs and susceptibilities of individual allograft recipients. Accordingly, there is a need in the art to identify other factors that can lead to improved selection and treatment of kidney allograft recipients.

### SUMMARY OF THE INVENTION

In one aspect, the present invention provides novel methods, primer sets and kits for determining whether a subject has or is predisposed to early rejection of a kidney allograft that is associated with an IL-1 family member polymorphism. In one embodiment, the method comprises determining a genotype in an IL-1 family member gene of the subject, and in particular whether the subject's nucleic acids contain an allele (or marker) selected from the group consisting of: an A1,A2 allele at an IL1A VNTR locus; an A2 allele at an IL-1A+4845 locus; an E2 allele at an IL-1B3953 locus; and combinations of the aforesaid alleles.

An allele associated with increased risk of early kidney allograft rejection can be detected by any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Amplification methods are selected from the group consisting of: the polymerase chain reaction (PCR), the ligase chain reaction (LCR), strand displacement amplification (SDA), cloning, and variations of the above (e.g. RT-PCR and allele specific amplification). Oligonucleotides necessary for amplification may be selected, for example, from within the IL-1 gene loci, either flanking the marker of interest (as required for PCR amplification) or directly overlapping the marker (as in ASO hybridization). In one embodiment, the sample is hybridized with a set of primers, which hybridize 5′ and 3′ in a sense or antisense sequence to the vascular disease associated allele, and is subjected to a PCR amplification.

An allele comprising a polymorphism in an IL-1 family member may also be detected indirectly, e.g. by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications.

One embodiment of the invention provides sets of primers for use in determining a genotype in an IL-1 family member gene of a subject. The inventive primer sets may contain one or more oligonucleotides, including 5′ and 3′ oligonucleotides that hybridize 5′ and 3′ to at least one allele of an IL-1 locus haplotype. PCR amplification oligonucleotides should hybridize between 25 and 2500 base pairs apart, preferably between about 100 and about 500 bases apart, in order to produce a PCR product of convenient size for subsequent analysis. Primers for use in the diagnostic method(s) and kits of the invention include SEQ ID NOs. 1 through 18 and subsets thereof, for example, SEQ ID NOs. 1 through 6.

In another aspect, the invention features kits for performing the above-described assays, the kits comprising oligonucleotides (including natural and/or synthetic compositions such as synthetic oligonucleotides, restriction fragments, cDNAs, synthetic peptide nucleic acids (PNAs), and the like) useful in the identification of polymorphisms in IL-1 family member alleles. The inventive kits may comprise sets of primers as described above; optionally, labeled oligonucleotides may be included in the kits, to allow ease of identification in the assays. The kit can include a nucleic acid sample collection means and a means for determining an IL-1 family member genotype. The kit may also contain a control sample or samples (either positive or negative controls or both) or a standard, and/or a device for assessing the results. In other embodiments, the kit comprises additional reagents and components including: DNA amplification reagents, DNA polymerase, nucleic acid amplification reagents, restriction enzymes, buffers, a nucleic acid sampling device, DNA purification device, deoxyribonucleotides, oligonucleotides (e.g. probes and primers) and other reagents or components known in the art.

As described above, the control may be a positive or negative control. Further, the control sample may contain the positive (or negative) products of the allele detection technique employed. For example, where the allele detection technique is PCR amplification, followed by size fractionation, the control sample may comprise DNA fragments of the appropriate size. Likewise, where the allele detection technique involves detection of a mutated protein, the control sample may comprise a sample of mutated protein. However, it is preferred that the control sample comprises the material to be tested. For example, the controls may be a sample of genomic DNA or a cloned portion of the IL-1 gene cluster. Preferably, however, the control sample is a highly purified sample of genomic DNA where the sample to be tested is genomic DNA.

The oligonucleotides present in said kit may be used for amplification of the region of interest or for direct allele specific oligonucleotide (ASO) hybridization to the markers in question. Thus, the oligonucleotides may either flank the marker of interest (as required for PCR amplification) or directly overlap the marker (as in ASO hybridization).

Information obtained using the assays and kits described herein (alone or in conjunction with information on another genetic defect or environmental factor, which contributes to the disease or condition that is associated with the presence of a certain allele or alleles for an IL-1 family member) is useful for determining whether a non-symptomatic subject is likely to develop the particular disease or, in particular, early rejection of a kidney allograft. The test results will assist in assessing the risk profile for the occurrence of acute allograft rejection episodes prior to the transplant itself. As a result, the information can allow a more customized approach to medical prophylaxis of rejection of the transplant(s). For example, this information can enable a clinician to more effectively prescribe a therapy that will address risk assessment of the disease or condition on a molecular basis.

In one embodiment, the information would be used to alter treatment to prevent early rejection of a kidney allograft. For example, patients at relatively high risk for early kidney allograft rejection can be treated with stronger induction immunotherapy to prevent rejection. Another alternative would be to select more potent maintenance immunosuppressive agents or increase the dose of the maintenance agent(s) to offset the higher risk of rejection that would be anticipated if the standard therapy was instituted.

The information of risk assessment can also be used to identify relatively low-risk patients that might be suitable for minimization of typical maintenance immunotherapy. For low-risk patients, the strategy of induction therapy can be eliminated, and/or the particular maintenance agents and doses reduced or eliminated without the consequence of early rejection.

### DETAILED DESCRIPTION OF THE INVENTION

Advances in molecular biology techniques have led to relatively simple and inexpensive genetic screening methods that make it possible to determine the genotype of an individual, and identify alleles (polymorphisms) that are associated with certain disease states, either causally or indirectly, via linkage to a gene or genes that are causally involved with the disease state. The ability to identify patients who are afflicted with or predisposed to a particular disease state facilitates the development of prophylactic or therapeutic treatments that are tailored to individual patients, and thereby maximizes the benefit of treatment while minimizing any risk associated therewith.

Specimens to be Tested

Nucleic acid to be used for the determination of genotype can be obtained from any specimen that contains nucleic acid, including fresh or frozen whole blood (with or without common anticoagulants such as citrate, EDTA or heparin), white blood cells (i.e., obtained from buffy coat, bone marrow, or other source) or other body fluids. DNA can also be isolated from specimens collected with swabs (for example, buccal, pharyngeal, eye, or other swabs), from washed cells from urine, saliva or other bodily fluid, fresh or frozen stool, or tissue samples (for example, muscle, liver, heart, brain, kidney or other tissue collected by biopsy or other means). Blood may collected onto absorbent paper and dried to provide a source of nucleic acid. When a treated paper such as FTA®-treated paper (Whatman Inc. Clifton, N.J.) is used, the cells are lysed and high molecular weight DNA is immobilized within the matrix.

Alternatively, nucleic acid tests can be performed on dry samples (e.g. hair or skin). When using RNA or protein, the cells or tissues that may be utilized must express an IL-1 gene. Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR In Situ Hybridization: Protocols and Applications, Raven Press, N.Y.).

As used herein, the term “nucleic acid” refers to polynucleotides or oligonucleotides such as deoxyribonucleic acid (DNA), and, where appropriate, ribonucleic acid (RNA). The term also comprehends, as equivalents, analogs of either RNA or DNA made from nucleotide analogs (e.g. peptide nucleic acids) and as applicable to the embodiment being described, single (sense or antisense) and double-stranded polynucleotides.

In one embodiment of the invention, blood cells are obtained from the patient by finger prick, and collected on absorbent paper. In a further embodiment, the blood is collected using an AmpliCARD Sampling Kit (Chemicon International, Inc., Temecula, Calif.). Nucleic acid (i.e., DNA) is then isolated from the dried blood spots and analyzed; target sequences may be amplified using the polymerase chain reaction (PCR) and oligonucleotide primers that target the specific polymorphic DNA region within the gene.

Numerous commercially available kits are available to facilitate specimen collection and processing, including DNeasy®), RNeasy®, FlexiGene™, PaxGene™ and QUIAamp® (Quiagen Inc., Valencia Calif.), MagExtractor® (Toyobo Co. Ltd., Osaka, Japan), and Nucleon™ kits (Scotlab Ltd., Coatbridge, UK). Those of ordinary skill in the art of genotyping are aware of these kits and their use. Moreover, numerous protocols for genotypic analysis are also know in the art, and described, for example, in Current Protocols in Human Genetics (John Wiley & Sons, Inc., New York, N.Y.; including an online version; 2002).

Detection of Alleles

Many methods for detecting specific alleles at polymorphic loci are known in the art and disclosed in the aforementioned references; the preferred method(s) will depend, in part, upon the molecular nature of the polymorphism. For example, in single nucleotide polymorphisms (or SNPs), which are major contributors to genetic variation comprising some 80% of all known polymorphisms, the various allelic forms of the polymorphic locus differ by a single base-pair. SNPs tend to occur in two different forms (although up to four different forms, corresponding to the four different nucleotide bases occurring in DNA, are theoretically possible), meaning that they can be genotyped by a simple plus/minus assay rather than a length measurement, making them amenable to automated analysis. Additionally, SNPs are mutationally more stable than other polymorphisms, enhancing their utility in studies of linkage disequilibrium between markers and an unknown variant that are done to map disease-causing mutations.

As disclosed in U.S. Pat. No. 6,268,142, the relevant disclosure of which is hereby incorporated by reference, a variety of methods are available for detecting the presence of a particular single nucleotide polymorphic allele in an individual. Such techniques include dynamic allele-specific hybridization (DASH), microplate array diagonal gel electrophoresis (MADGE), pyrosequencing, oligonucleotide-specific ligation, the TaqMan® system (Applied Biosystems, Foster City, Calif.), and various DNA “chip” technologies (such as GeneChip® arrays, and related reagents and equipment available from Affymetrix Inc., Santa Clara, Calif.). These methods require amplification of the target genetic region, typically by PCR. Other methods, based on the generation of small signal molecules by invasive cleavage followed by mass spectrometry or immobilized padlock probes and rolling-circle amplification, eliminate the need for PCR. Several of the methods known in the art for detecting specific single nucleotide polymorphisms are summarized below. The method of the present invention is understood to include all available methods.

Numerous methods of analyzing SNPs are known in the art. For example, the SNP can be detected by using a specialized exonuclease-resistant nucleotide that is only incorporated into a primer when it is complementary to the target molecule, as disclosed, e.g., in Mundy, C. R. (U.S. Pat. No. 4,656,127). Another example is discussed in Cohen, et al. (French Patent 2,650,840; PCT Appln. No. WO91/02087), which discloses a solution-based method for determining the identity of the nucleotide of a polymorphic site using a primer that is complementary to allelic sequences immediately 3′ to the polymorphic site. An alternative method, known as Genetic Bit Analysis or GBA™, described by Goelet et al. (PCT Appln. No. 92/15712), uses mixtures of labeled terminators and a primer that is complementary to the sequence 3′ to a polymorphic site. The method of Goelet et al. is preferably a heterogeneous phase assay, in which the primer or the target molecule is immobilized to a solid phase.

Additional assays that are known in the art include primer-guided nucleotide incorporation procedures for assaying polymorphic sites in DNA (Komher et al., Nucl. Acids. Res. 17:7779, 1989; Sokolov, B. P., Nucl. Acids Res. 18:3671, 1990; Syvanen et al., Genomics 8:684, 1990; Kuppuswamy et al., Proc. Natl. Acad. Sci. USA 88:1143, 1991; Prezant et al., Hum. Mutat. 1:159, 1992; Ugozzoli et al., GATA 9:107, 1992; Nyren, P. et al., Anal. Biochem. 208:171, 1993; Syvanen et al., Amer. J. Hum. Genet. 52:46, 1993; van't Wout et al., AIDS 12:1169, 1998). Mutations that produce premature termination of protein translation can be evaluated using the protein truncation test (PTT; Roest et. al., Hum. Mol Genet. 2:1719, 1993; van der Luijt et. al., Genomics 20:1, 1994; Den Dunnen J T and Van Ommen G J, Hum. Mutat. 14:95, 1999).

In addition to methods that focus primarily on the detection of one nucleic acid sequence, profiles may also be assessed in such detection schemes. Fingerprint profiles may be generated, for example, by utilizing a differential display procedure, Northern analysis and/or RT-PCR. One detection method is allele specific hybridization using probes overlapping a region of at least one allele and having about 5, 10, 20, 25, or 30 nucleotides around the mutation or polymorphic region. In one embodiment of the invention, several probes capable of hybridizing specifically to other allelic variants are attached to a solid phase support, e.g., a “chip” (which can hold up to about 250,000 oligonucleotides).

Oligonucleotides can be bound to a solid support by a variety of processes, including lithography. Mutation detection analysis using these chips comprising oligonucleotides, also termed “DNA probe arrays” is described e.g., in Cronin et al., Hum. Mutat. 7:244, 1996; Freeman et al., Biotechniques 29:1042, 2000; Cuzin M, Transfus. Clin. Biol. 8:291, 2001. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli et al., Proc. Natl. Acad. Sci. USA 87:1874, 1990), transcriptional amplification system (Kwoh et al., Proc. Natl. Acad. Sci. USA 86:1173, 1989), and Q-Beta Replicase (Lizardi et al., Bio/Technology 6:1197, 1988). Amplification products may be assayed in a varety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different primers that are differentially labeled and thus can each be differentially detected. Hybridization based detection means allow the differential detection of multiple PCR products in a sample. Other techniques are known in the art to allow multiplex analyses of a plurality of markers.

In one embodiment, the method includes the steps of (i) collecting a sample of cells from an individual, (ii) isolating nucleic acid (e.g., genomic, mRNA or both) from the cells, (iii) contacting the nucleic acid with one or more primers which specifically hybridize 5′ and 3′ to at least one allele of an IL-1 family member under conditions such that hybridization and amplification of the allele occurs, and (iv) detecting the amplification product. These detection schemes are especially useful for the detection of nucleic acid molecules if such molecules are present in very low numbers. In another embodiment of the subject assay, the allele of the IL-1 family member is identified by alterations in restriction enzyme cleavage patterns. For example, sample and control DNA is isolated, amplified (optionally), digested with one or more restriction endonucleases, and fragment length sizes are determined by gel electrophoresis.

In another embodiment, any of a variety of sequencing reactions known in the art can be used to directly sequence the allele. Exemplary sequencing reactions include those based on techniques developed by Maxim and Gilbert (Proc. Natl. Acad Sci USA 74:560, 1977) or Sanger (Proc. Nat. Acad. Sci USA 74:5463, 1977). A variety of automated sequencing procedures may be utilized when performing the subject assays (see, for example Naeve et al., Biotechniques 19:448, 1995), including sequencing by mass spectrometry (see, for example PCT publication WO 94/16101; Cohen et al., Adv Chromatogr 36:127, 1996; and Griffin et al., Appl Biochem Biotechnol 38:147, 1993). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and piperidine) can be used to detect mismatched bases in RNA/RNA or RNA/DNA or DNA/DNA heteroduplexes (Myers, et al., Science 230:1242, 1985). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. After digestion of the mismatched regions, the resulting material is then separated by size on denaturing polyacrylamide gels to determine the site of mutation. See, for example, Cotton et al. (Proc. Natl. Acad Sci USA 85:4397, 1988), and Saleeba et al. (Methods Enzymol. 217:286, 1992). The control DNA or RNA can be labeled for detection.

In still another embodiment, the mismatch cleavage reaction employs one or more proteins that recognize mismatched base pairs in double-stranded DNA (so called “DNA mismatch repair” enzymes, for example, the mutY enzyme of E. coli or the thymidine DNA glycosylase from HeLa cells; see Hsu et al., Carcinogenesis 15:1657, 1994). According to one such embodiment, a probe based on an allele of an IL-1 locus haplotype is hybridized to a cDNA or other DNA product from a test cell(s), treated with a DNA mismatch repair enzyme, and the cleavage products, if any, can be detected from electrophoresis protocols or the like (see, for example, U.S. Pat. No. 5,459,039).

In other embodiments, alterations in electrophoretic mobility will be used to identify an IL-1 locus allele. For example, single strand conformation polymorphism (SSCP) may be used to detect differences in electrophoretic mobility between mutant and wild type nucleic acids (Orita et al., Proc Natl. Acad. Sci USA 86:2766, 1989; Cotton, R G, Mutat Res 285:125, 1993; and Hayashi, Genet Anal Tech Appl 9:73, 1992). Such methods may further utilize heteroduplex analysis to separate double stranded heteroduplex molecules on the basis of changes in electrophoretic mobility (Keen et al., Trends Genet 7:5, 1991). The movement of alleles in polyacrylamide gels containing a gradient of denaturant may also be assayed using denaturing gradient gel electrophoresis (DGGE) (Myers et al., Nature 313:495, 1985). In a further embodiment, a temperature gradient is used in place of a denaturing agent gradient to identify differences in the mobility of control and sample DNA (Rosenbaum and Reissner, Biophys Chem 265:12753, 1987).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al., Nature 324:163, 1986; Saiki et al, Proc. Natl. Acad. Sci USA 86:6230, 1989). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization; see Gibbs et al., Nucleic Acids Res. 17:2437, 1989) or at the extreme 3′ end of one primer where, under appropriate conditions, mismatch can prevent, or reduce polymerase extension (see Prossner, Tibtech 11:238, 1993). In addition it may be desirable to introduce a novel restriction site in the region of the mutation to create cleavage-based detection (Gasparini et al., Mol. Cell Probes 6: 1, 1992). Amplification may also be performed using Taq ligase for amplification (Barany, Proc. Natl. Acad. Sci USA 88:189,1991).

In another embodiment, identification of the allelic variant is carried out using an oligonucleotide ligation assay (OLA), as described, e.g., in U.S. Pat. No. 4,998,617 and in Landegren, U. et al., Science 241:1077, 1988). Nickerson, et al. have described a nucleic acid detection assay that combines attributes of PCR and OLA (Proc. Natl. Acad. Sci. USA 87:8923, 1990). In this method, PCR is used to achieve the exponential amplification of target DNA, which is then detected using OLA. Several techniques based on this OLA method have been developed and can be used to detect alleles of an IL-1 locus haplotype; see, for example, U.S. Pat. No. 5,593,826; To be et al., Nucleic Acids Res 24: 3728, 1996). OLA combined with PCR permits typing of two alleles in a single microtiter well and can facilitate high throughput if used with reagents that lead to the production of two different colors.

Primers and Kits

Another embodiment of the invention is directed sets of primers useful in determining a genotype in an IL-1 family member gene (and kits comprising such primers). Determining the genotype of an individual using such primers (or sets thereof) facilitates detecting a predisposition for early rejection of a kidney allograft in the individual. The inventive primer sets may contain one or more oligonucleotides, including 5′ and 3′ oligonucleotides that hybridize 5′ and 3′ to at least one allele of an IL-1 locus haplotype. PCR amplification oligonucleotides should hybridize between 25 and 2500 base pairs apart, preferably between about 100 and about 500 bases apart, in order to produce a PCR product of convenient size for subsequent analysis. Primers for use in the diagnostic method(s) and kits of the invention include SEQ ID NOs. 1 through 18, and subsets thereof, for example, SEQ ID NOs. 1 through 6.

The design of additional oligonucleotides for use in the amplification and detection of IL-1 polymorphic alleles by the method of the invention is facilitated by the availability of both updated sequence information from human chromosome 2q13—which contains the human IL-1 locus, and updated human polymorphism information available for this locus. For example, the DNA sequences for IL-1A, IL-1B and IL-1RN are publicly available under GenBank Accession No. X03833, GenBank Accession No. X04500 and GenBank Accession No. X64532, respectively. Suitable primers for the detection of a human polymorphism in these genes can be readily designed using this sequence information and standard techniques known in the art for the design and optimization of primers sequences. Optimal design of such primer sequences can be achieved, for example, by the use of commercially available primer selection programs such as Primer 2.1, Primer 3 or GeneFisher (See also, Nicklin et al., Genomics 19: 382, 1995; Nothwang et al. Genomics 41:370, 1997; Clark, et al., Nucl. Acids. Res., 14:7897, 1986 [published erratum appears in Nucleic Acids Res. 15:868, 1987], and the Genome Database (GDB) project at the URL http://www.gdb.org).

The invention further provides kits that are useful in analyzing or genotyping patient samples at IL-1 family member alleles, comprising oligonucleotides (including natural and/or synthetic compositions such as synthetic oligonucleotides, restriction fragments, cDNAs, synthetic peptide nucleic acids (PNAs), and the like) useful in the identification of polymorphisms in IL-1 family member alleles. The assay kits and/or methods may employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

The kit may, optionally, also include nucleic acid sampling means. Nucleic acid sampling means are well known to one of skill in the art and can include, but are not limited to, substrates (such as filter papers or other specimen collection matrices); nucleic acid purification reagents, lysis buffers, proteinase solutions and the like; PCR reagents, such as 10× reaction buffers, thermostable polymerase, dNTPs, and the like; and allele detection means such as certain restriction enzymes, allele specific oligonucleotides, and/or degenerate oligonucleotide primers for nested PCR from dried blood. The inventive kits may further comprise one or more control samples comprising one or more specific alleles in the IL-1 family region. Processes for using these kits are also comprehended within the invention.

The following examples are offered by way of illustration, and not by way of limitation. Those skilled in the art will recognize that variations of the invention embodied in the examples can be made, especially in light of the teachings of the various references cited herein, the disclosures of which are incorporated by reference.

### EXAMPLE 1

A total of 87 kidney allograft recipients was evaluated. Of these patients, 14 underwent early rejection of the transplanted kidney (defined as rejection within 21 days of transplant), whereas 73 did not. The patient population is described in Table 1 below.

TABLE 1

| | Early | Early | | |
|---|---|---|---|---|
| | Rejection+ | Rejection− | Stat | P value |
| Age N | 14 | 73 | T = 0.01 | 0.9955 |
| Mean | 43.5 | 43.5 | | |
| S.D. | 12.2 | 12.4 | | |
| Min-Max | 22-62 | 15-63 | | |
| Gender N | 14 | 73 | X² = 1.69 | 0.1934 |
| Female | 3 (21%) | 29 (40%) | | |
| male | 11 (79%) | 44 (60%) | | |
| Race N | 14 | 73 | | |
| Caucasion | 12 (86%) | 43 (59%) | | |
| Black | 1 (7%) | 13 (18%) | | |
| Hispanic | 1 (7%) | 11 (15%) | | |
| Other | 0 (0%) | 6 (8%) | | |
| # Prev. Tx. N | 14 | 73 | | |
| None | 14 (100%) | 69 (95%) | | |
| One | 0 (0%) | 3 (4%) | | |
| Two | 0 (0%) | 1 (1%) | | |
| Dialysis N | 14 | 73 | | |
| None | 2 (14%) | 13 (18%) | | |
| Hemo | 11 (79%) | 45 (62%) | | |
| Peritoneal | 1 (7%) | 15 (21%) | | |
| Duration of Dialysis N | 14 | 73 | t = 0.43 | 0.6692 |
| Mean | 21.4 | 18.4 | | |
| S.D. | 24.8 | 24.4 | | |
| Min.-Max. | 0-93 | 0-168 | | |
| LPRA N | 14 | 73 | t = 2.77 | 0.0071 |
| Mean | 0.6 | 5.0 | Wilcoxon | 0.2214 |
| S.D. | 1.1 | 13.1 | X² = 0.80 | 0.6399 |
| Min.-Max. | 0-3 | 0-83 | Fisher's | 0.6088 |
| Median | 0.0 | 0.0 | | |
| IQ Range | 0.0, 2.0 | 0.0, 3.0 | | |
| <20 | 14 (100%) | 69 (95%) | | |
| 20 | 0 (0%) | 4 (5%) | | |
| MPRA N | 14 | 73 | t = 2.41 | 0.0183 |
| Mean | 0.7 | 6.1 | Wilcoxon | 0.1618 |
| S.D. | 1.7 | 18.7 | X² = 1.02 | 0.3131 |
| Min.-Max. | 0-6 | 0-98 | Fisher's | 0.5876 |
| Median | 0.0 | 0.0 | | |
| IQ Range | 0.0, 0.5 | 0.0, 3.5 | | |
| <20 | 14 (100%) | 68 (93%) | | |
| 20 | 0 (0%) | 5 (7%) | | |
| Donor N | 14 | 73 | X² = 2.14 | 0.1436 |
| LRD | 6 (43%) | 34 (47%) | | |
| Cadaver | 3 (21%) | 26 (36%) | | |
| LURD | 5 (36%) | 13 (18%) | | |

Specimens (peripheral blood) were obtained from allograft recipients and DNA was isolated and analyzed for various alleles in the IL-1 region of the human genome, as described in Table 2 below.

TABLE 2

| Polymorphism | Reference(s) | Observations | Alleles |
|---|---|---|---|
| IL-1A VNTR | Bailly et al., Mol | Analyze by gel | A1: 803 bp |
| IL1 alpha intron 6 | Immunol. 33: 999, 1996 | electrophoresis; size | A2: 1217 bp |
| (46 bp VNTR) | | will depend on | A3: 757 bp |
| | | number of tandem | A4: 941 bp |
| | | repeats (TR), varying | A5: 619 bp |
| | | from 527 bp for 3 TR | A6: 1079 bp |
| | | to 1217 bp for 18 TR | A7: 665 bp |
| | | | A8: 1171 bp |
| | | | A9: 1033 bp |
| | | | A10: 527 bp |
| | | | A11: 1125 bp |
| | | | A12: 849 bp* |
| IL-1A +4845 | Armitage et al., J Periodontol | Digest with Fnu4H1 | A1: G |
| IL1 alpha, SNP at +4845 | 71: 164, 2000 | yields 124 bp and | A2: T |
| | | 29 bp fragments, or | |
| | | 153 bp uncut | |
| | | fragment, depending | |
| | | on allele(s) present, | |
| | | as well as 76 bp | |
| | | fragment with both. | |
| IL-1B +3953 | Pociot et al., Eur J Clin | Digest with Taq1 | E1: C |
| IL-1 beta exon 5, | Invest 22: 396, 1992; | yields 135 bp and | E2: T |
| SNP at +3953, Taq | Guasch et al., Cytokine | 114 bp cut fragments, | |
| RFLP | 8: 598, 1996; Cantagrel et | or 249 bp uncut | |
| | al., Arthritis & | fragment, depending | |
| | Rheumatism 42: 1093, | on allele(s) present | |
| | 1999; Conti et al., Am. J. Path. | | |
| | 157: 1685, 2000; El- | | |
| | Omar et al., Nature | | |
| | 404: 398, 2000 | | |
| IL-1B −511 | di Giovine et al, Hum. | Digest with Ava1 | A1: C |
| IL-1 beta promoter, | Mol. Genet. 1: 450, 1992; | yields 190 bp and | A2: T |
| SNP at −511, Ava1 | Cantagrel et al supra; | 114 bp cut fragments, | |
| RFLP | Conti et al., supra; El- | or 304 bp uncut | |
| | Omar et al., supra | fragment, depending | |
| | | on allele(s) present | |
| IL-1RN VNTR | Cantagrel et al supra; | Analyze by gel | A1: 442 bp |
| IL1ra intron 2, 86 bp | Conti et al., supra; El- | electrophoresis; size | A2: 270 bp |
| VNTR | Omar et al., supra | of bands will depend | A3: 528 bp |
| | | on number of TR, | A4: 356 bp |
| | | varying from 270 bp | A5: 614 bp |
| | | for 2 TR to 614 bp | |
| | | for 6 TR. | |

*Alleles that include 13 repeats (987 bp), 11 repeats (895 bp) or 7 repeats (711 bp) are also possible, but have not been identified.

Genotyping analysis was performed using PCR based amplification of the isolated DNA, as described in Table 3 below.

TABLE 3

| Polymorphism | Primer | PCR Conditions | Reaction | |
|---|---|---|---|---|
| IL-1A VNTR | #40284 | 95° C. 15 min, 62° C. 1 min, | H₂0 | 12.8 μl |
| | (SEQ ID | 72° C. 1.5 min, 1×; | 10 × HotStarTaq ® Buffer | 2.0 μl |
| | NO: 1) | 94° C. 1 min, 62° C. 1 min, | 5 mM dNTPs (0.2 mM) | 0.8 μl |
| | #40285 | 72° C. 1.5 min, 29×; | Oligo #40284 80 ng | 1.9 μl |
| | (SEQ ID | 72° C. 5 min, 1× | Oligo #40285 80 ng | 1.5 μl |
| | NO: 2) | | Template 50 ng | 0.5 μl |
| | | | HotStarTaq ® 5 u/μl | 0.5 μl |
| | | | Total volume | 20 μl |
| IL-1A +4845 | #43749 | 95° C. 15 min, 1×; | H₂0 | 28.9 μl |
| | (SEQ ID | 94° C. 1 min, 56° C. 1 min, | 10 × HotStarTaq ® Buffer | 5.0 μl |
| | NO: 3) | 72° C. 2 min, 39×; | 5 mM dNTPs (0.2 mM) | 2.0 μl |
| | #43750 | 72° C. 5 min, 1× | Oligo #43749 80 ng | 1.0 μl |
| | (SEQ ID | | Oligo #43750 80 ng | 2.6 μl |
| | NO: 4) | | Template 100 ng | 10.0 μl |
| | | | HotStarTaq ® 5 u/μl | 0.5 μl |
| | | | Total volume | 50 μl |
| IL-1B +3953 | #41448 | 95° C. 30 sec, 55° C. 30 sec, | H₂0 | 31.1 μl |
| | (SEQ ID | 72° C. 30 sec, 40× | 10 × AmpliTaq ® buffer | 5.0 μl |
| | NO: 5) | | 5 mM dNTPs (0.2 mM) | 2.0 μl |
| | #41449 | | Oligo #41448 50 ng | 0.65 μl |
| | (SEQ ID | | Oligo #41449 50 ng | 0.74 μl |
| | NO: 6) | | Template 100 ng | 10.0 μl |
| | | | AmpliTaq ® 5 u/μl | 0.5 μl |
| | | | Total volume | 50 μl |
| IL-1B −511 | #41450 | Same as for IL-1B +3953 | H₂0 | 31.1 μl |
| | (SEQ ID | | 10 × AmpliTaq ® buffer | 5.0 μl |
| | NO: 7) | | 5 mM dNTPs (0.2 mM) | 2.0 μl |
| | #41451 | | Oligo #41448 50 ng | 0.70 μl |
| | (SEQ ID | | Oligo #41449 50 ng | 0.70 μl |
| | NO: 8) | | Template 100 ng | 10.0 μl |
| | | | AmpliTaq ® 5 u/μl | 0.5 μl |
| | | | Total volume | 50 μl |
| IL-1RN VNTR | #42057 | Same as for IL-1A VNTR | H₂0 | 11.4 μl |
| | (SEQ ID | | 10 × HotStarTaq ® buffer | 2.0 μl |
| | NO: 9) | | 5 mM dNTPs (0.2 mM) | 0.8 μl |
| | #42058 | | Oligo #42057 80 ng | 3.0 μl |
| | (SEQ ID | | Oligo #42058 80 ng | 1.8 μl |
| | NO: 10) | | Template 50 ng | 0.5 μl |
| | | | HotStarTaq ® 5 u/μl | 0.5 μl |
| | | | Total volume | 20 μl |

Controls included human IL-18 binding protein (GenBank accession number AF110799; Novick et al., Immunity 10:127, 1999) utilizing the primers shown as SEQ ID NOs.: 13 and 14; and TIGIRR (GenBank accession number AF284436; Born et al., J. Biol. Chem. 275:29946, 2000) utilizing the primers shown as SEQ ID NOs.: 15 and 16 for the Ava1 digest, and SEQ ID NOs.: 17 and 18 for the Taq 1 digest. Results are shown in Table 4 below.

TABLE 4

| | Early | | | |
|---|---|---|---|---|
| | Rejection+ | Early Rejection− | Stat | P value |
| IL-1A VNTR | | | | |
| N | 13 | 72 | X² = 8.58 | 0.0034 |
| A2, A1 | 6 (46%) | 9 (13%) | | |
| Other | 7 (54%) | 63 (88%) | | |
| IL-1A +4845 | | | | |
| N | 13 | 72 | X² = 6.063 | 0.0100 |
| A2 | 11 (85%) | 33 (46%) | | |
| Other | 2 (15%) | 39 (54%) | | |
| IL-1B +3953 | | | | |
| N | 13 | 72 | X² = 4.998 | 0.0255 |
| E2 | 9 (69%) | 26 (36%) | | |
| Other | 4 (31%) | 46 (64%) | | |
| IL-1B −511 | | | | |
| N | 13 | 72 | | |
| C, C | 4 (31%) | 27 (38%) | | |
| C, T | 6 (46%) | 34 (47%) | | |
| T, T | 3 (23%) | 11 (11%) | | |
| IL-1RN VNTR | | | | |
| N | 13 | 72 | X² = 2.14 | 0.1436 |
| A2 | 12 (92%) | 53 (74%) | | |
| Other | 1 (8%) | 19 (26%) | | |

These results indicate that there is a statistically significant difference in the frequency of certain alleles at three loci (IL-1A VNTR, IL-1A+4845 and IL-1B+3953) between patients that rapidly reject kidney allografts (defined as rejection within 21 days of transplant) and those that do not.

| | |
|---|---|
| SEQ ID NO: | Sequence |

| | |
|---|---|
| 1 | gcctctagactcatagaacttagtc |
| 2 | gtgaggtcaggccattgcattg |
| 3 | atggttttagaaatcatcaagcctagggca |
| 4 | aatgaaaggaggggaggatgacagaaatgt |
| 5 | gttgtcatcagactttgacc |
| 6 | ttcagttcatatggaccaga |
| 7 | tggcattgatctggttcatc |
| 8 | gtttaggaatcttcccactt |
| 9 | cccctcagcaacactcc |
| 10 | ggtcagaagggcagaga |
| 11 | ttggaggatggcccatgaagacc |
| 12 | ctgttacgcgcccggatgaaaaa |
| 13 | tcgtcactctcctggtcag |
| 14 | cacgcatgtgacctcagg |
| 15 | tcacatgcgtggtggtgg |
| 16 | tggaggtgcataatgccaag |
| 17 | tggaggtgcataatgccaag |
| 18 | agaagacgttcccctgctg |

1. A method of identifying an individual predisposed to early rejection of a kidney allograft, comprising determining a genotype of an IL-1 family member gene, wherein the presence of one or more alleles selected from the group consisting of:
(a) an A1,A2 allele combination at an IL1A VNTR intron 6 locus;
(b) an A2 allele at an IL-1A+4845 locus; and
(c) an E2 allele at an IL-1B+3953 locus indicates that the individual is at increased risk for early rejection of the kidney allograft.

2. The method of claim 1, wherein the genotype is determined by amplifying nucleic acid from the IL-1 family member gene.

3. The method of claim 2, wherein the nucleic acid is amplified using a primer selected from the group consisting of any of SEQ ID NOs. 1 through 6.

4. A primer set for use in determining a genotype in an IL-1 family member gene, selected from the group consisting of:
(a) SEQ ID NO:1 and SEQ ID NO:2;
(b) SEQ ID NO:3 and SEQ ID NO:4;
(c) SEQ ID NO:5 and SEQ ID NO:6;
and combinations thereof.

5. (canceled).

6. (canceled).

7. (canceled).

8. A method for treating a patient predisposed to early kidney allograft rejection, said method comprising:
(a) determining a genotype in an IL-1 family member gene, wherein the presence of one or more alleles, selected from the group consisting of an A1,A2 allele combination at an IL1A intron 6 VNTR locus; an A2 allele at the IL-1A+4845 locus; and an E2 allele at the IL-1B+3953 locus, indicates that the individual is at increased risk for early rejection of the kidney allograft; and
(b) altering adjusting immunosuppressive therapy in the patient.

9. (canceled).

10. (canceled).
